(19) United States
(12) Patent Application Publication
(10) Pub. No.: US 2006/0206199 A1
(43) Pub. Date: Sep. 14, 2006

(54) ANEURYSM TREATMENT DEVICES

(76) Inventors: Stacey D. Churchwell, Twinsburg, OH (US); Bruce E. Hammer, Minnetonka, MN (US); Jodi Prosise, Forest Lake, MN (US); Andrew T. Schieber, St. Louis Park, MN (US); Charles L. Euteneuer, St. Michael, MN (US)

Correspondence Address: WILSON SONSINI GOODRICH & ROSATI, 650 PAGE MILL ROAD, PALO ALTO, CA, 94304-1050, US

(21) Appl. No.: 11/276,224

(22) Filed: Feb. 17, 2006

Related U.S. Application Data
(63) Continuation-in-part of application No. 11/275,455, filed on Jan. 5, 2006.
(60) Provisional application No. 60/661,647, filed on Mar. 12, 2005.

(51) Int. Cl. A61F 2/06

(52) U.S. Cl. 623/1.25

(57) ABSTRACT

Devices for treating aneurysms are disclosed. The devices are adapted and configured to modify blood flow at the aneurysm. More specifically, the invention discloses devices for treating cerebral aneurysms using devices adapted and configured to be delivered to a blood vessel in the brain on a distal tip of a microcatheter. The aneurysm devices comprise: a device adapted to be delivered to a blood vessel aneurysm on a distal tip of a catheter and further adapted to modify blood flow at the aneurysm.

### CROSS-REFERENCE

This application is a continuation-in-part of application of Ser. No. 11/275,455, filed Jan. 5, 2006 by Stacey D. Churchwell et al. entitled Aneurysm Treatment Devices and Methods which is incorporated herein by reference in its entirety and to which priority is claimed under 35 U.S.C. § 120.

This application also claims the benefit of U.S. Provisional Application No. 60/661,647, filed Mar. 12, 2005 by Stacey D. Churchwell et al. entitled Aneurysm Neck Occlusion Disc, which is incorporated herein by reference in its entirety.

### BACKGROUND OF THE INVENTION

An aneurysm is an abnormal widening or ballooning of a portion of an artery, related to weakness in the wall of the artery or blood vessel. Some common locations for aneurysms include: the aorta; the brain (cerebral); the legs (popliteal artery aneurysm); the intestine (mesenteric artery); and the spleen.

Aneurysms are either congenital (present before birth) or acquired. It is thought that defects in some component(s) of the artery wall may be responsible for aneurysms. Although in some instances, high blood pressure is thought to be a contributing factor. Atherosclerotic disease (cholesterol buildup in arteries) may also contribute to the formation of certain types of aneurysms. As a result of a defect in the artery wall, the aneurysm can rupture, which can result in profuse bleeding.

Like other aneurysms, cerebral aneurysms may occur as a congenital defect or may develop later in life. One type of cerebral aneurysm is the berry aneurysm, which can be over 2 cm in size. The berry aneurysm resembles a sack of blood attached to one side of the blood vessel and typically has a narrow neck. Other types of aneurysms involve widening or dilation of the entire circumference of a blood vessel in an area. Still other types appear as a ballooning out of a part of a blood vessel. It is estimated that 5% of the population has some type of aneurysm in the brain, with up to 10% of those affected having more than one aneurysm. The vessel wall of an aneurysm can be as thin as 15-100 microns. Cerebral aneurysms can rupture and cause bleeding or hemorrhaging in the area between the brain and the surrounding membrane (the arachnoid); or can extend into the subarachnoid space. Fortunately, most aneurysms under ¼ inch in diameter do not rupture. However, aneurysms that do rupture can have serious consequences including stroke and death. Approximately 20,000 people in the United States suffer a subarachnoid hemorrhage each year. An estimated 1 to 2 percent (three to six million) of Americans have cerebral aneurysms. Although they can occur at any age, they are slightly more common in adults than children and are slightly more common in women than men. One treatment for cerebral aneurysm involves opening the skull and clipping the aneurysm to stop further bleeding.

Other devices and methods for treating aneurysms include: U.S. Pat. Nos. 5,980,514 to Kupiecki et al. for Aneurysm Closure Device Assembly; U.S. Pat. No. 6,096,034 to Kupiecki et al. for Aneurysm Closure Device Assembly; U.S. Pat. No. 6,183,495 to Lenker et al. for Wire Frame Partial Flow Obstruction Device for Aneurysm Treatment; U.S. Pat. No. 6,551,303 to Van Tassel et al. for Barrier Device for Ostium of Left Atrial Appendage; U.S. Pat. No. 6,569,190 to Whalen II et al. for Methods for Treating Aneurysms; U.S. Pat. No. 6,663,607 to Slaikey et al. for Bioactive Aneurysm Closure Device Assembly and Kit; U.S. Pat. No. 5,782,905 to Richter for Endovascular Device for Protection of Aneurysm; U.S. Pat. No. 5,951,599 to McCrory for Occlusion System for Endovascular Treatment of An Aneurysm; U.S. Pat. No. 6,063,111 to Hieshima et al. for Stent Aneurysm Treatment System and Method; U.S. Pat. No. 6,093,199 to Brown et al. for Intra-Luminal Device for Treatment of Body Cavities and Lumens and Method of Use; U.S. Pat. No. 6,168,622 to Mazzocchi for Method and Apparatus for Occluding Aneurysms; U.S. Pat. No. 6,626,928 to Raymond et al. for Occlusion Device for Treating Aneurysm and Use Therefore; U.S. Pat. No. 6,746,468 to Sepetka et al. for Devices and Methods for Treating Vascular Malformations; U.S. Pat. No. 6,802,851 to Jones et al. for Stent Aneurysm Embolization Method Using Collapsible Member and Embolic Coils; U.S. Pat. No. 6,855,153 to Saadat for Embolic Balloon; U.S. Pat. No. 6,860,899 to Rivelli Jr. for Method for Treating Neurovascular Aneurysms; U.S. Pat. No. 6,036,720 to Abrams et al. for Sheet Metal Aneurysm Neck Bridge; U.S. Pat. No. 6,139,654 to Teoh for Minimally Occlusive Flow Disruptor Stent for Bridging Aneurysm Necks; U.S. Pat. No. 5,935,148 to Villar et al. for Detachable, Varying Flexibility, Aneurysm Neck Bridge; U.S. Pat. No. 6,379,329 to Naglreiter et al. for Detachable Balloon Embolization Device and Method; U.S. Pat. No. 4,638,803 to Rand for Medical Apparatus for Inducing Scar Tissue Formation in a Body; U.S. Pat. No. 5,476,472 to Dormandy Jr. et al. for Embolization Device and Apparatus Including an Introducer Cartridge and A Delivery Catheter and Method for Delivering the Embolization Device; U.S. Pat. No. 5,746,734 to Dormandy Jr. et al. for Introducer Cartridge for Delivering an Embolization Device; U.S. Pat. No. 5,571,171 to Barone et al. for Method for Repairing An Artery in a Body; and U.S. Patent Publications 2003/0018294 to Cox for Aneurysm Treatment Device and Method of Use; 2004/0044391 to Porter for Device for Closure of a Vascular Defect and Method of Treating the Same; 2004/0059407 to Escamilla et al. for Expandable Stent and Delivery System; 2004/0078071 to Escamilla et al. for Expandable Stent with Radiopaque Markers and Stent Delivery System; 2004/0111112 to Hoffman for Method and Apparatus for Retaining Embolic Material; 2004/0193206 to Gerberding et al. for Methods and Devices for the Treatment of Aneurysms; 2004/0193246 to Ferrera for Method and Apparatus for Treating Aneurysms and Other Vascular Defects; 2005/0033409 to Burke et al. for Aneurysm Treatment Device and Method of Use; 2002/0143349 to Gifford III et al. for Devices and Methods for Treating Vascular Malformations; 2002/0133190 to Horton et al. for InSitu Formable and Self-Forming Intravascular Flow Modifier (IFM), Catheter and IFM Assembly, and Method for Deployment of Same; 2002/0198592 to Wallace et al. for Intracranial Stent and Method of Use; 2003/0100945 to Yodfat et al. for Implantable Intraluminal Device and Method of Using Same in Treating Aneurysm; 2003/0109917 to Rudin for Stent Vascular Intervention Device and Method; 2003/0139802 to Wulfman et al. for Medical Device; 2003/0204244 to tiger for Aneurysm Exclusion Stent; 2005/0107823 to Leone et al. for Anchored Stent and Occlusive Device for Treatment of Aneurysms; 2005/0119684 to Guterman et al. for Aneurysm Buttress Arrangement; 2005/0133046 to Becker et al. for Compositions and Methods for Improved Occlusion of Vascular Defects; European Patent Application EP 1616585 A1 to Tijsma for Device for the Treatment of Aneurysms.

### SUMMARY OF THE INVENTION

The invention discloses devices and methods for treating aneurysms. The devices are adapted and configured to modify blood flow at the aneurysm. More specifically, the invention discloses devices and methods for treating cerebral aneurysms using a device adapted and configured to be delivered to a blood vessel in the brain on a distal tip of a microcatheter.

In one embodiment of the invention, an aneurysm treatment device is disclosed. The aneurysm device comprises: an implant adapted to be delivered to a blood vessel aneurysm on a distal tip of a catheter and further adapted to modify blood flow at the aneurysm. In some embodiments, the device also includes an inflatable balloon. In such embodiments, the device can comprise one or more struts adapted to anchor the balloon relative to the aneurysm. Alternatively, the device can comprise a disc for anchoring the device after deployment. The struts or disc can be adapted to engage an interior surface of a blood vessel or engage a stent positioned within the interior of the blood vessel. The struts and/or disc can be provided with surface texturing, perforations, or other modifications.

In other embodiments, the aneurysm treatment device comprises an expandable disk. The expandable disk can be configured to provide a support and a patch. In some embodiments, the expandable disk is adapted to engage a stent. In other embodiments, the disk can be provided with hooks adapted to anchor at least a portion of the disk along a wall of the vessel. In other embodiments, one or more threads can be provided to retain the disk with fluidic pressure within the lumen of the vessel. In still other embodiments, the disk can be retained in position relative to an opening of an aneurysm using a ring adapted to be positioned within the vessel. A spiral wire can also be adapted to support the membrane. In still other embodiments, the implant comprises an umbrella comprised of struts and a membrane supporting the membrane.

The aneurysm treatment devices of the invention can be further adapted to partially occlude a neck of the aneurysm.

The invention also includes kits for treating a blood vessel aneurysm. Kits according to the invention comprise: an aneurysm treatment device adapted to be delivered on a distal end of a catheter to a blood vessel aneurysm and further adapted to modify blood flow at a neck of the aneurysm; and a catheter. The kit can further comprise a stent.

The invention also includes a method for treating a blood vessel aneurysm. A method according to the invention includes: accessing a vasculature; advancing a catheter adapted to engage an aneurysm treatment device at a distal tip through the vasculature to reach the aneurysm; and deploying the aneurysm treatment device from the distal tip of the catheter at the aneurysm to modify blood flow at the aneurysm. In some embodiments of the method, a stent can be deployed within the vasculature adjacent the aneurysm. Further, the step of anchoring the aneurysm treatment device to the stent can be performed. In other methods, the aneurysm treatment device can be anchored to the wall of the blood vessel and/or can be anchored to a stent. The method of the invention can result in partially occluding a neck of the aneurysm and/or modifying the blood flow in an aneurysm.

### INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-B depict a blood vessel having an aneurysm therein; the aneurysm of FIG. 1A has a wide neck opening into the lumen of the blood vessel, while the aneurysm of FIG. 1B has a narrow neck opening into the lumen of the blood vessel;

FIGS. 2A-D depict an aneurysm treatment device adapted and configured to be delivered on a distal tip of a catheter and further adapted to modify blood flow;

FIGS. 2E-G depict an aneurysm treatment device adapted and configured to be delivered on a distal tip of a catheter and further adapted to modify blood flow similar to the device depicted in FIGS. 2A-D wherein the balloon anchors the device and a disc modifies blood flow;

FIGS. 3A-E depicts an alternate embodiment of an aneurysm treatment device adapted and configured to be delivered on a distal tip of a catheter and further adapted to modify blood flow;

FIGS. 4A-C depict yet another alternate embodiment of an aneurysm treatment device adapted and configured to be delivered on a distal tip of a catheter and further adapted to modify blood flow;

FIGS. 5A-C depict still another embodiment of an aneurysm treatment device adapted and configured to be delivered on a distal tip of a catheter and further adapted to modify blood flow;

FIGS. 6A-C depict yet another embodiment of an aneurysm treatment device adapted and configured to be delivered on a distal tip of a catheter and further adapted to modify blood flow;

FIGS. 7A-D depict devices with anchoring mechanisms suitable for use with the embodiments of the invention;

FIGS. 8A-B depict a catheter suitable for use in delivering any of the aneurysm treatment devices of the invention and the tip of the catheter engaging an aneurysm treatment device of the invention; and

FIG. 9 is a flow chart illustrating a method of the invention.

### DETAILED DESCRIPTION OF THE INVENTION

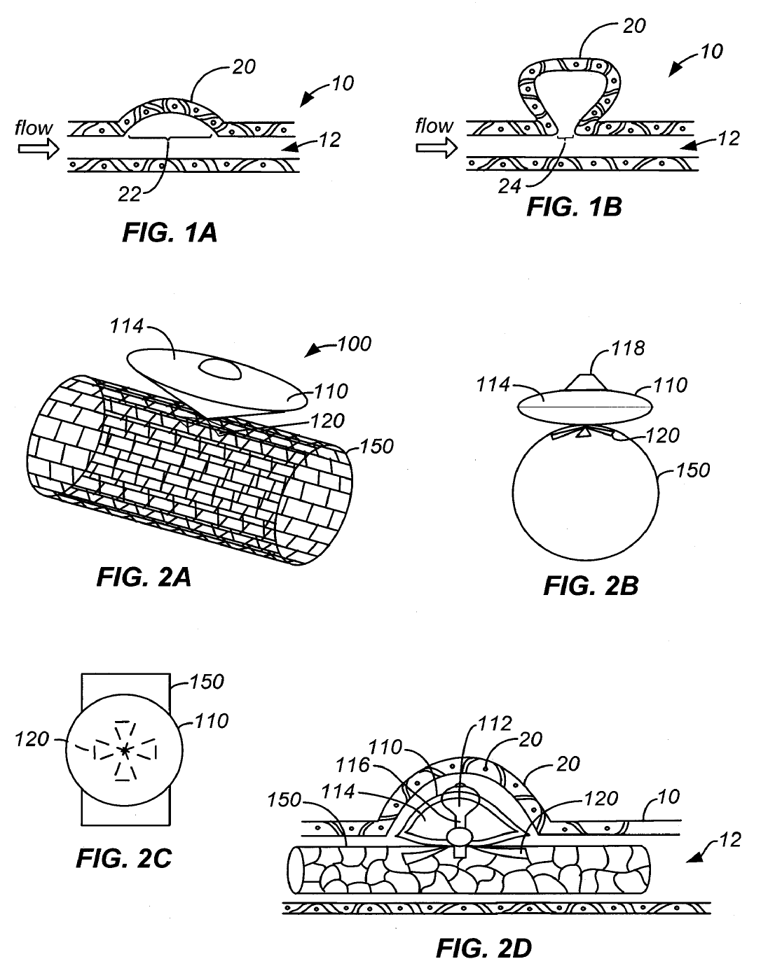

FIGS. 1A-B depict a blood vessel 10 defining a lumen 12 having an aneurysm 20 therein. The aneurysm 20 of FIG. 1A has a wide neck opening 22 into the lumen 12 of the blood vessel 10. In contrast, the aneurysm 10 of FIG. 1B has a narrow neck 24 opening into the lumen 12 of the blood vessel 10.

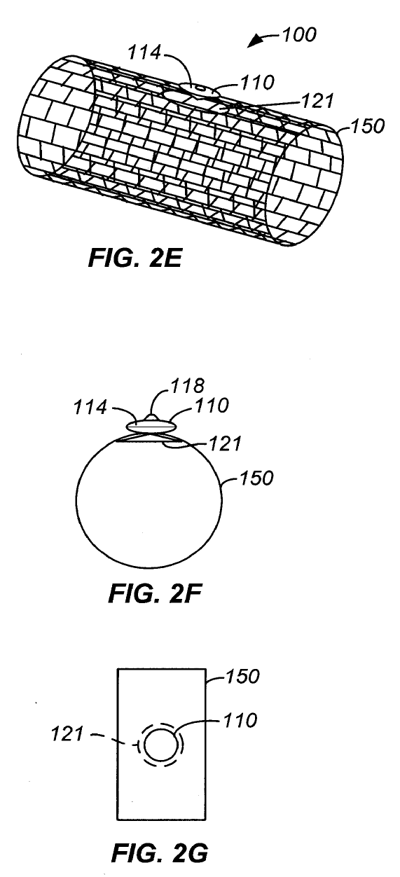

FIGS. 2A-D depict an embodiment of an aneurysm treatment device 100 according to the invention adapted and configured to be delivered on a distal tip of a catheter and further adapted to modify blood flow. The aneurysm treatment device 100 comprises balloon 110 which is attached to a retainer clip or anchoring mechanism 120. The balloon 110 can be flat, disk-shaped balloon. The anchoring mechanism enables the device 100 to attach to, for example, a stent 150. The balloon 110 has one or more valves 112 contained in its lumen which communicate with the interior lumen 114 of the balloon 110 through a valve port 116. The valves and valve port enable the balloon to be inflated or deflated by a user. A tip 118 is provided, which can be tapered, that is formed of a material of sufficient strength and stiffness that it is suitable for use in advancing the device over a guide wire through the vasculature. As depicted in FIG. 2D, the balloon can be inflated to fit within the aneurysm 20. Once inflated, the device 100 modifies the blood flow within the aneurysm by filling the aneurysm neck. Filling, or substantially filling, the aneurysm neck with the device 100 decreases the blood flow within the aneurysm, thereby permitting the remaining blood in the aneurysm to coagulate and decreases the amount of pressure present on the vessel walls of the aneurysm, and decreasing the likelihood that the aneurysm will rupture.

The anchoring mechanism 120 illustrated with respect to the device depicted in FIGS. 2-D is formed from two or more wings. The wings are positioned such that the wings radiate away from an axis of the device from which the device would be secured to a retaining apparatus, for example, a stent. As depicted, the axis is a central axis which corresponds to the longitudinal axis of the valve mechanism 112. The wings are adapted to be positioned within the lumen of the stent through one or more apertures (e.g. through the woven surface of the stent) to be positioned within the lumen. Once positioned within the lumen, the wings enable the device to be secured to the stent, while retaining the ability to pivot about the central axis. The wings may be positioned parallel to the surface of the balloon or substantially parallel to the surface of the balloon.

Turning to the embodiment shown in FIGS. 2E-G, an alternative embodiment of an aneurysm treatment device 100 is depicted. The device is adapted and configured to be delivered on a distal tip of a catheter and further adapted to modify blood flow. As with the device depicted in FIG. 2A, the aneurysm treatment device 100 comprises balloon 110 which is attached to a retainer clip or anchoring mechanism 121. The balloon 110 can be flat, disk-shaped balloon. The disc 121 is provided that assists the device 100 is securing to, for example, a stent 150. The balloon 110 has one or more valves 112 contained in its lumen which communicate with the interior lumen 114 of the balloon 110 through a valve port 116. The valves and valve port enable the balloon to be inflated or deflated by a user. A tip 118 is provided, which can be tapered, that is formed of a material of sufficient strength and stiffness that it is suitable for use in advancing the device over a guide wire through the vasculature. The balloon 110 in this embodiment serves the anchor the device to the stent. The balloon can be inflated such that it fits within at least a portion of the aneurysm 20, similar to the depiction in FIG. 2D. However, because the balloon in this embodiment primarily secures the device, it need only be sized to enable the device to be secured to the stent.

Once inflated, the device 100 is positioned relative to the aneurysm such that the disc 121 modifies the blood flow within the aneurysm by covering all or a portion of the aneurysm neck. Covering, or substantially covering, the aneurysm neck with the disc 121 decreases the blood flow within the aneurysm, thereby permitting the remaining blood in the aneurysm to coagulate and decreases the amount of pressure present on the vessel walls of the aneurysm, and decreasing the likelihood that the aneurysm will rupture.

As discussed and illustrated in FIGS. 2E-G, the anchoring mechanism 121 is depicted as a disc. Similar to the mechanism of FIG. 2A the disc is positioned such that the disc radiates away from an axis of the device. The disc can be formed such that it has a solid layer. Alternatively, the disc can be formed such that it has perforations on its surface (e.g., holes, slits, etc.). The perforations can be arranged in a pattern or randomly. Additionally, with any of the configurations surface features can be provided. For example, the surface can be textured or provided with a layer of biocompatible material to enhance biological acceptance of the device. As will be appreciated by those skilled in the art, other portions of the device can be textured or coated to enhance the performance of the device. Alternate configurations and orientations of securing the device can be employed without departing from the invention.

Additionally, as illustrated in FIGS. 2E-2G, the diameter or span of the device in a plane is such that the disc 121 has a diameter greater than the balloon 110. In contrast, the diameter or span of the device in a plane for the embodiment illustrated in FIGS. 2A-D, is such that the diameter formed by the wings 120 in a plane s less than the diameter formed by the balloon 110 in the same plane. As will be appreciated by those skilled in the art, other diameters can be used without departing from the scope of the invention such that, for example, the diameter of the disc is less than or equal to the diameter of the balloon, or the diameter of the wings is greater than or equal to the diameter of the balloon.

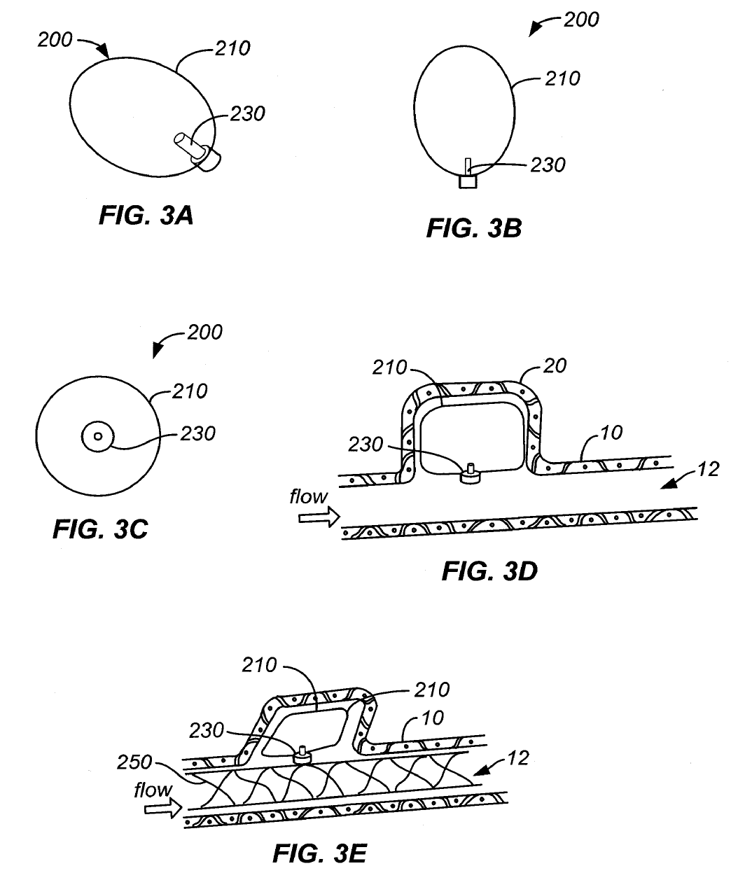

FIGS. 3A-E depicts an alternate embodiment of an aneurysm treatment device 200 also adapted and configured to be delivered on a distal tip of a catheter and further adapted to modify blood flow. The device 200 comprises a conformable balloon 210 with a valve 230. The valve 230 can be used to inflate and deflate the balloon 210. The valve can be a two-valve system, similar to the device depicted in FIG. 2, or can be a single valve design that is potentially flow directed. When inflated, the balloon 210 is positioned within an aneurysm in order to fill, or substantially fill, the aneurysm neckwith the device to decrease the blood flow and thereby permitting the remaining blood in the aneurysm to coagulate and decreases the amount of pressure present on the vessel walls of the aneurysm,. As illustrated in FIG. 3E, the device 200 can be anchored to a stent 250 that is positioned within the lumen 12 of the vessel 10. The valve 230 can be configured to secure to the stent 250 or can be provided with additional anchoring mechanisms, such as the retainer clip depicted in FIG. 2. Upon inflation, the balloon can assume a shape that corresponds to the shape of the aneurysm neck or can assume a predetermined shape, e.g., a shape dictated by the balloon pre-form

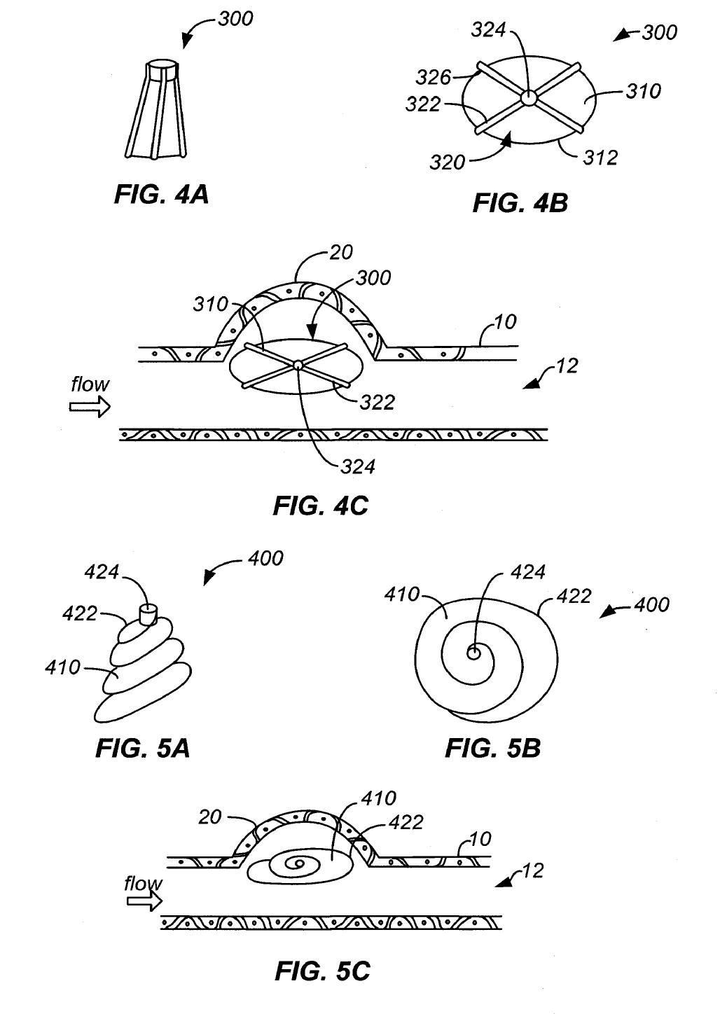

Turning now to FIGS. 4A-C, another embodiment of an aneurysm treatment device 300 adapted and configured to be delivered on a distal tip of a catheter and further adapted to modify blood flow is depicted. In this embodiment, the device 300 comprises a membrane support frame 320 that includes a central joint 324 having one or more arms 322 adapted to extend a membrane 310 away from central joint 324 in order to achieve a disc, or substantially disc-like, profile. Additionally, a wire can be provided in edge 312 of the membrane to provide further support of the membrane 310 once it assumes the disc profile. As illustrated in FIG. 4C, the device can be deployed to partially occlude a neck of an aneurysm and secured in place with barbs 326 located on the ends of arms 322. The device 300 can also be deployed with a stent (not shown) as discussed in other embodiments. The membrane support frame 320 can be configured from shape memory alloy, e.g. nitinol, to facilitate the device 300 expanding into the disc shape. The device remains deployed by the use of one or more securement mechanism, such as those described below. Once deployed, the device is not removed.

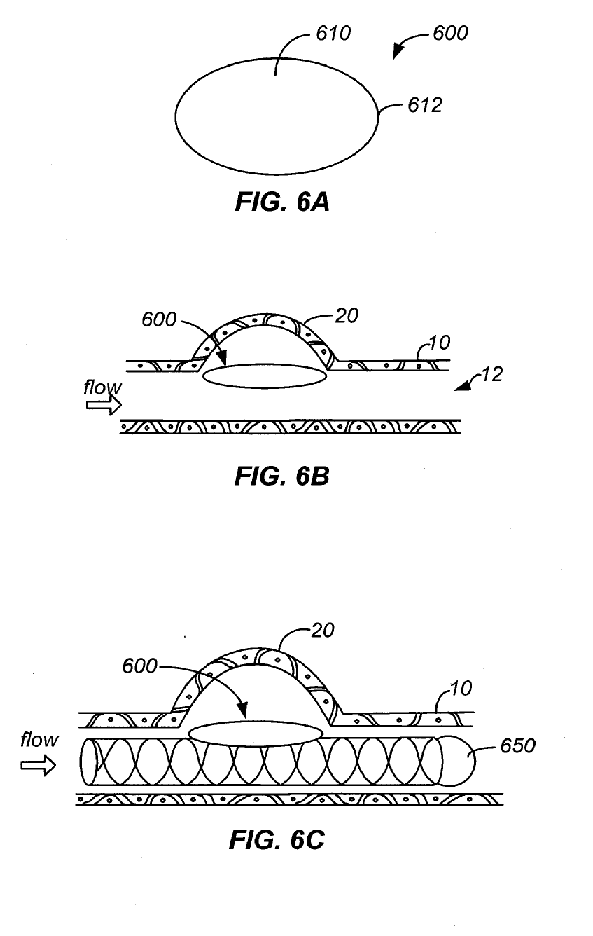

FIGS. 5A-C depict still another embodiment of an aneurysm treatment device 400 adapted and configured to be delivered on a distal tip of a catheter and further adapted to modify blood flow. The aneurysm treatment device 400 has a central catheter attachment fitting 424 from which extends a helical support 422, such as a wire. The helical support could take a variety of configurations, including, but not limited to, a double helix. During deployment, the device 400 can achieve a small diameter profile suitable for advancing the device through the vasculature on the distal tip of a catheter. Once the device 400 is positioned adjacent an aneurysm 20 the device 400 flattens into a disc, as illustrated in FIG. 5B. Similar to the device shown in FIG. 4C, the device 400 can be deployed to partially occlude a neck of an aneurysm 20, as depicted in FIG. 5C. The device remains deployed by use of one or more securement or anchoring mechanisms, such as those described below.

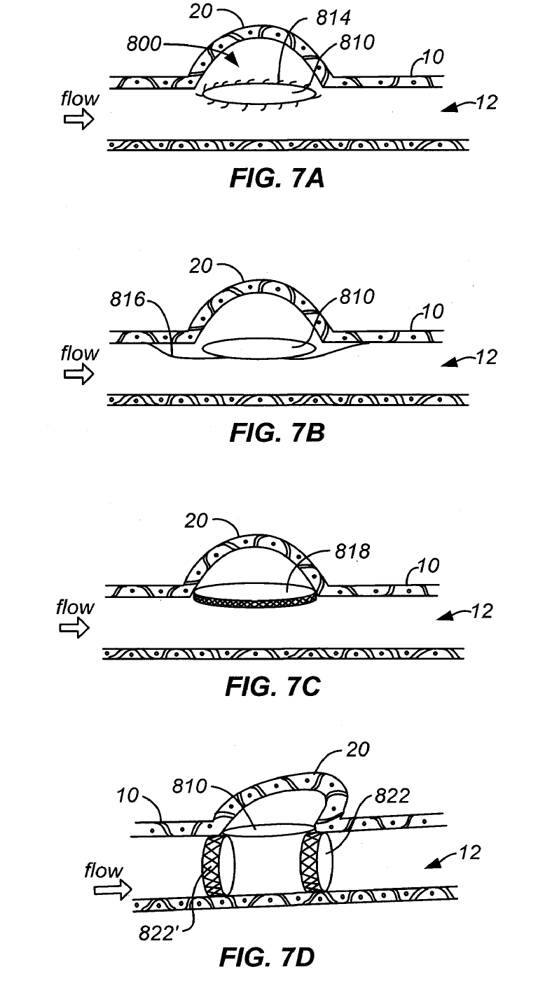

FIGS. 6A-C depict yet another embodiment of an aneurysm treatment device 600 adapted and configured to be delivered on a distal tip of a catheter and further adapted to modify blood flow. The device comprises an expandable disk 610 which can feature a wire 612 around the perimeter. As shown in FIG. 6B the device is 600 is deployed in the blood vessel 10 and is positioned relative to the aneurysm 12. A neural stent 650 is deployed to secure the device in place.

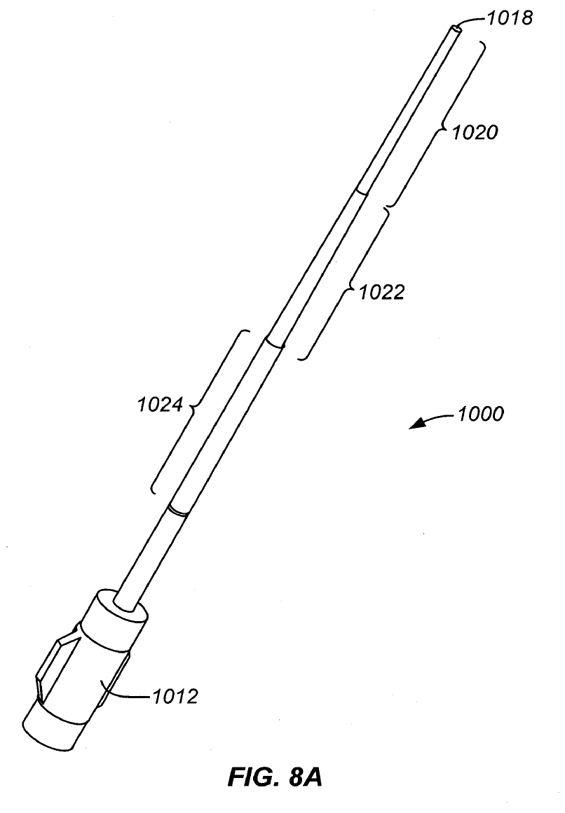

FIGS. 7A-D depict anchoring or securement mechanisms suitable for use with an aneurysm treatment device 800 adapted and configured to be delivered on a distal tip of a catheter and further adapted to modify blood flow. For purposes of illustration, the device 800 comprises a disk 810 and a plurality of hooks 814 around the perimeter of the disk. The hooks 814 facilitate anchoring the device into position with respect to the aneurysm 20 as shown in FIG. 7A. In an alternative embodiment, the device 800 can be configured to provide threads of biocompatible material 816 that extend from the device 800 through the lumen 12 to engage the walls of the blood vessel. The threads 816 are held into position against the walls of the blood vessel 10 as a result of fluidic pressure, e.g. pressure resulting from the flow of blood through the lumen. As depicted in FIG. 6B the device 900 can have a cage structure 822 around the perimeter of the disk 810 that facilitates maintaining patency of the disk when it is deployed. The cage 822 can also be used to keep the device in position relative to the aneurysm. Alternatively the cage, which can be formed like a stent, can be deployed first, with the membrane forming the disk deployed subsequent to positioning of the cage. Finally, with respect to FIG. 7D one or two small rings 822, or stent-like structures, can be positioned in the lumen 12 adjacent the aneurysm 20. The disk 810 is then deployed from one of the rings and, if the second ring is present, secured to the second ring 822′.

In operation, the aneurysm treatment devices disclosed herein operate by altering or modifying the flow of blood to or within an aneurysm by positioning the device relative to the neck of an aneurysm such that the device covers at least a portion of the neck of the aneurysm.

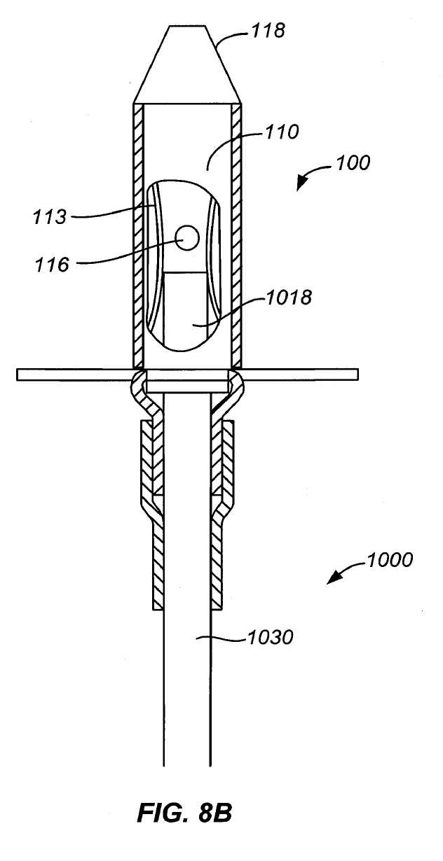

FIGS. 8A-B depict a microcatheter 1000 suitable for use in delivering any of the aneurysm treatment devices of the invention and the tip of the catheter engaging an aneurysm treatment device, such as device 100 depicted in FIG. 2D of the invention. The microcatheter 1000 of FIG. 8A has a Luer connector 1012 at a proximal end. A tip 1018 is provided at the distal end which is adapted to engage the aneurysm treatment devices of the invention. The microcatheter 1000 can be configured to have regions of flexibility, such that the distal region 1020 has a flexibility that is different from a mid section 1022, and which is different from a proximal section 1024. More detailed information on the configurations of catheters is contained in U.S. Pat. No. 6,355,027 to Le et al. for Flexible Microcatheter; U.S. Pat. No. 6,733,487 to Keith et al. for Balloon Catheter with Distal Guide Wire Lumen; U.S. Pat. No. 6,663,660 to Dusbabek et al. for Stent Delivery System Having Stent Securement Apparatus; and U.S. Pat. No. 6,610,069 to Euteneuer et al. for Catheter Support for Stent Delivery.

Turning now to FIG. 8B, a microcatheter 1000 is depicted in combination with an aneurysm treatment device, such as device 100 depicted in FIG. 2D. The device 100 is adapted to receive the tip 1018 of the microcatheter 1000. A retainer constraining tube 1002 is provided that enables the catheter 1000 to release the stent retainer upon deployment of the device from the tip of the delivery catheter. The device 1000 has been depicted with a portion of the balloon 110 cut away to expose the interior valve body 113 and valve port 116. When the microcatheter 1000 engages the device 100, a continuous lumen extends from the microcatheter tip through the valve port into the balloon. Material can then been injected into the valve body 113 where it is released into the balloon of the device. The balloon can be inflated with sterile water, saline solution, or contrast media. As long as the balloon is attached to the catheter tip, fluid can pass into and out of the valve port. Once the balloon has achieved a desired profile, it can be disengaged from the catheter tip.

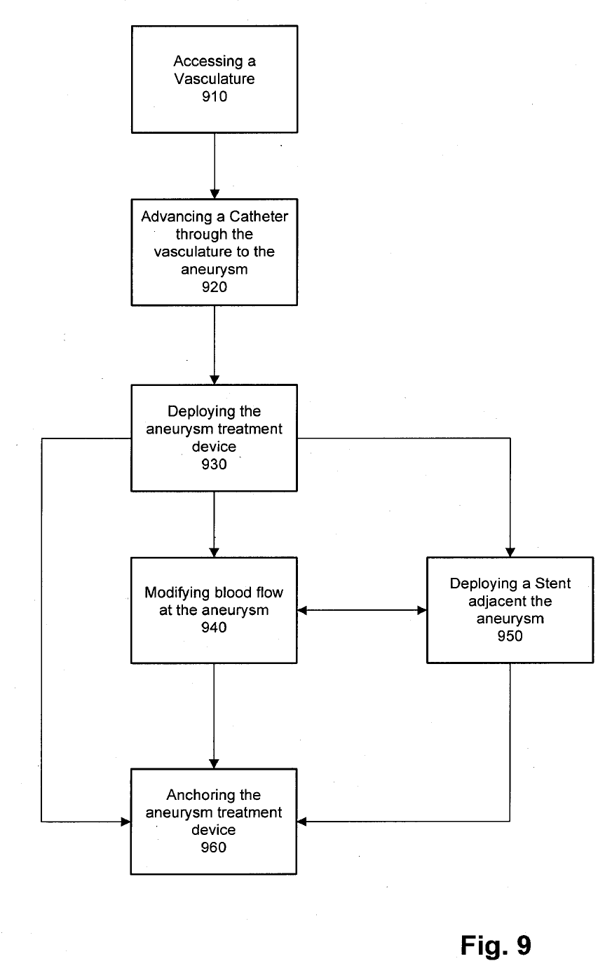

FIG. 9 is a flow chart showing the method steps of the invention. The devices disclosed herein are inserted into a catheter in collapsed form. The end of the catheter is maneuvered into the neck of the aneurysm. For the embodiments shown in FIGS. 2-5, the device can be secured at the distal end of a microcatheter and advanced through the vasculature to the aneurysm. Once positioned at the aneurysm, the device is deployed to modify the blood flow at the aneurysm. The device can be secured in place using a variety of mechanisms, as described above. Alternatively, the device need not be secured at the distal end of the catheter before advancing through the vasculature, i.e., the device can be pushed without being secured.

For embodiments shown in FIGS. 4-7, once the end of the catheter is maneuvered into the neck of the aneurysm the disc can then be pushed out into the aneurysm neck and the memory material in the disc restores the shape of the device to a deployed/pre-defined shape. At this point, if the device was secured to the catheter, the disc is then disconnected from, e.g. from a guidewire, using a mechanical, electrochemical or chemical mechanism. Barbs then anchor the disc in the desired position, e.g., into the wall of an aneurysm neck.

As illustrated in FIG. 9, the invention also includes a method for treating a blood vessel aneurysm. The method includes: accessing a vasculature 910; advancing a catheter adapted to engage an aneurysm treatment device at a distal tip through the vasculature to reach the aneurysm 920; and deploying the aneurysm treatment device from the distal tip of the catheter at the aneurysm to modify blood flow at the aneurysm 930. In some embodiments of the method, a stent can be deployed 950 within the vasculature adjacent the aneurysm. Further, the step of anchoring the aneurysm treatment device 960 to the stent can be performed. In other methods, the aneurysm treatment device can be anchored to the wall of the blood vessel and/or can be anchored to a stent. The method of the invention can result in partially occluding a neck of the aneurysm and/or modifying the blood flow in an aneurysm 940. As will be appreciated by those skilled in the art, the order of the steps of the method can be varied without departing from the scope of the invention. For example, after deploying the aneurysm treatment device 930, modification of the blood flow can occur at the aneurysm 940. Alternatively, concurrently, or prior to the step of modifying the blood flow, a stent can be deployed adjacent the aneurysm 950. An additional alternative could be the step of anchoring the aneurysm treatment device 960 following the step of deploying the aneurysm treatment device 930.

In accordance with the various embodiments of the present invention described herein, the mechanical supporting framework or device may be made from a variety of materials such as metal, composite, plastic or amorphous materials, which include, but are not limited to, steel, stainless steel, cobalt chromium plated steel, titanium, nickel titanium alloy (nitinol), super elastic alloy, and polymethylmethacrylate. The supporting framework or device may also include other polymeric materials that are biocompatible and provide mechanical strength, that include polymeric material with ability to carry and delivery therapeutic agents, that include bioabsorbable properties, as well as composite materials and composite materials of titanium and polyetheretherketone (PEEK), composite materials of polymers and minerals, composite materials of polymers and glass fibers, composite materials of metal, polymer, and minerals.

Where a portion of the device includes nitinol, the shape of the device may be dynamically modified using thermal, electrical or mechanical manipulation. For example, the nitinol device or supporting framework may be expanded or contracted once deployed.

Candidate materials for the devices and components would be known by persons skilled in the art and include, for example, suitable biocompatible materials such as metals (e.g. stainless steel, shape memory alloys, such a nickel titanium alloy nitinol) and engineering plastics (e.g. polycarbonate). See, for example U.S. Pat. Nos. 5,190,546 to Jervis for Medical Devices Incorporating SIM Memory Alloy Elements and U.S. Pat. No. 5,964,770 to Flomenblit for High Strength Medical Devices of Shape Memory Alloy. In one embodiment, the outer exoskeleton may be made of materials such as titanium, cobalt chrome stainless steel. Alternatively, the membrane can be made of biocompatible polymers such as polyetheretherketone (PEEK), polyarylamide, polyethylene, silicone polyurethane, expanded poly tetraflouroethylene (ePTFE) and polysulphone.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An aneurysm treatment device comprising:
an implant adapted to be delivered to a blood vessel aneurysm on a distal tip of a catheter and further adapted to modify blood flow at the aneurysm.

2. The aneurysm treatment device of claim 1 wherein the implant comprises an inflatable balloon.

3. The aneurysm treatment device of claim 2 further comprising a strut adapted to anchor the balloon relative to the aneurysm.

4. The aneurysm treatment device of claim 3 wherein the strut is adapted to engage an interior surface of the blood vessel.

5. The aneurysm treatment device of claim 3 wherein the strut is adapted to engage a stent positioned within the blood vessel.

6. The aneurysm treatment device of claim 1 wherein the implant comprises an expandable disk.

7. The aneurysm treatment device of claim 6 wherein the expandable disk further comprises a support and a patch.

8. The aneurysm treatment device of claim 7 wherein the expandable disk is adapted to engage a stent.

9. The aneurysm treatment device of claim 6 wherein the expandable disk provides a plurality of hooks adapted to anchor at least a portion of the disk along a perimeter to a wall of a vessel.

10. The aneurysm treatment device of claim 6 wherein the disk has one or more threads adapted to retain the disk with fluidic pressure.

11. The aneurysm treatment device of claim 6 wherein the disk is retained in position relative to an opening of an aneurysm with a ring adapted to be positioned within a vessel.

12. The aneurysm treatment device of claim 1 wherein the implant comprises a spiral wire adapted to support a membrane.

13. The aneurysm treatment device of claim 1 wherein the implant comprises an umbrella comprised of struts and a membrane.

14. The aneurysm treatment device of claim 12 further comprising an anchor.

15. The aneurysm treatment device of claim 1 further adapted to partially occlude a neck of the aneurysm.

16. A kit for treating a blood vessel aneurysm comprising:
an aneurysm treatment device adapted to be delivered on a distal end of a catheter to a blood vessel aneurysm and further adapted to modify blood flow at a neck of the aneurysm; and
a catheter.

17. The kit of claim 16 further comprising a stent.
